(19) United States
(12) Patent Application Publication
(10) Pub. No.: US 2025/0312404 A1
(43) Pub. Date: Oct. 9, 2025

(54) COMPOSITION FOR IMPROVING UNIDENTIFIED COMPLAINTS, DEPRESSION, IMPAIRED CONCENTRATION, DECREASED MOTIVATION FOR SOCIALIZATION AND DAILY ACTIVITIES, AUTONOMIC NERVOUS SYSTEM DISORDERS, PAIN, CONTROL AND POOR SLEEP QUALITY ASSOCIATED WITH MENSTRUATION.

(71) Applicant: Tokiwa Phytochemical Co., Ltd., Sakura-shi, Chiba (JP)

(72) Inventors: Jinwei YANG, Sakura-shi, Chiba (JP); Tomoko KUNIYOSHI, Sakura-shi, Chiba (JP)

(21) Appl. No.: 18/577,877

(22) PCT Filed: Jul. 3, 2023
(86) PCT No.: PCT/JP2023/024654

§ 371 Date: Jan. 9, 2024

(51) Int. Cl. A61K 36/24; A61K 31/7048; A61P 15/08; A61P 25/00; A61P 25/20; A61P 25/24; A61P 29/00

(52) U.S. Cl. CPC A61K 36/24; A61K 31/7048; A61P 15/08; A61P 25/00; A61P 25/20; A61P 25/24; A61P 29/00; A61K 2236/333; A61K 2236/51; A61K 2236/53

(57) ABSTRACT

A composition having effects on improving unidentified complaints, depression, impaired concentration, decreased motivation for socialization and daily activities, autonomic nervous system disorders, pain, control and poor sleep quality associated with menstruation is provided. The composition comprises Apocynum venetum leaf extract for improving unidentified complaints, depression, impaired concentration, decreased motivation for socialization and daily activities, autonomic nervous system disorders, pain, control and poor sleep quality associated with menstruation.

### FIELD OF INVENTION

The present invention relates to a composition for improving unidentified complaints, depression, impaired concentration, decreased motivation for socialization and daily activities, autonomic nervous system disorders, pain, control and poor sleep quality associated with menstruation.

### BACKGROUND OF INVENTION

In recent years, the decline in the working population due to the super-aging society and declining birthrate has become a serious problem, prompting women's participation in the labor force. However, it has been noted that problems specific to women, such as unidentified complaints, premenstrual syndrome and menopausal symptoms associated with menstruation due to changes in female hormones, prevent women from being active in society. Providing appropriate support and measures to address these issues specific to women is expected to improve women's health.

Unidentified complaints associated with menstruation are believed to be related to fluctuations in female hormones. Estrogen (follicle hormone) and progesterone (luteal hormone) are secreted in large amounts during the luteal phase after ovulation. However, follicle and luteal hormone levels sharply drop in the second half of the luteal phase, causing a disturbance in the balance of hormones and neurotransmitters in the brain, which is believed to be the cause of unidentified complaints associated with menstruation.

Unidentified complaints associated with menstruation include a variety of symptoms, the main ones being pain (abdominal pain, headache, back pain and fatigue), autonomic nervous system disorders (hot flashes, loss of appetite or overeating and dizziness), negative emotions (emotional instability, irritability, depression and anxiety), impaired concentration, fluid retention (swelling, abdominal distension and breast tenderness) and poor sleep quality.

Meanwhile, Apocynum venetum L. (Luobuma, rafuma) belongs to the Apocynaceae family and is a perennial herb that grows wild in temperate regions from China to Europe and Asia. Apocynum venetum leaves are used as a tea in China and have been certified as a food for specified health uses in Japan for its proven blood pressure-lowering effects.

Regarding the stress-reducing and relaxing effects of Apocynum venetum, it has been reported that Apocynum venetum leaf extract may exhibit antidepressant (see, for example, Non-patent document 1 and Patent document 1) and anxiolytic effects (see, for example, Non-patent document 2) in animal studies. In addition, since Apocynum venetum leaf extract acts as an agonist of inhibitory neurotransmitter GABA receptors in the brain, clinical trials have shown that simultaneous intake of 25 mg of GABA and 25 mg of Apocynum venetum leaf extract per day reduces chromogranin A, an indicator of stress (see, for example, Non-patent document 3 and Patent document 2).

In recent years, the sleep-improving effects of Apocynum venetum leaves have also attracted attention. It was reported that a clinical trial in which subjects wore an electroencephalograph at night to evaluate their sleep using electroencephalograms showed that Apocynum venetum leaf extract was effective in increasing the percentage of non-REM sleep, which is deep sleep (see, for example, Non-patent document 4). Another clinical trial used the MA version of the OSA Sleep Questionnaire to evaluate how subjects experienced improved sleep and showed significant improvements in Factor II, which evaluates falling asleep and staying asleep, as well as in the effectiveness of resting and recovery from fatigue (see, for example, Non-patent document 5 and Patent document 3).

### PRIOR ART DOCUMENT

### Non-Patent Document

[Non-patent document 1] Butterweck V, Nishibe S, Sasaki T, Uchida M., “Antidepressant effects of apocynum venetum leaves in a forced swimming test.”, Biol Pharm Bull., 24 (7), 848-851 (2001).
[Non-patent document 2] Grundmann O, Nakajima J, Kamata K, Seo S, Butterweck V., “Kaempferol from the leaves of Apocynum venetum possesses anxiolytic activities in the elevated plus maze test in mice.”, Phytomedicine, 16, 295-302 (2009).
[Non-patent document 3] YAMATSU A, YAMASHITA Y, MARU I, YANG J, TATSUZAKI J and KIM M., “The Improvement of Sleep by Oral Intake of GABA and Apocynum venetum Leaf Extract.”, JOURNAL OF NUTRITIONAL SCIENCE AND VITAMINOLOGY 61 (2), 182-187 (2015).
[Non-patent document 4] Yamatsu A, Yamashita Y, Maru I, Yang J, Tatsuzaki J, Kim M., “The Improvement of Sleep by Oral Intake of GABA and Apocynum venetum Leaf Extract.”, J Nutr Sci Vitaminol (Tokyo), 61 (2), 182-187 (2015).
[Non-patent document 5] Nakata A, Yamashita S, Suzuki N, Liang T, Kuniyoshi T, Yang J, Takara T, “Effect of an Apocynum venetum Leaf Extract (VENETRON®) on Sleep Quality and Psychological Stress Improvement—A Randomized, Double-blind, Placebo-controlled Crossover Study—.”, Jpn Pharmacol Ther., 46 (1), 117-125 (2018).
[Patent document 1] Japanese Patent No. 4629933.
[Patent document 2] Japanese Patent Publication No. 2011-93842.
[Patent document 3] Japanese Patent No. 7099685.

### SUMMARY OF INVENTION

### Problems to be Solved by Invention

According to the “Survey on Health Promotion and Working Women 2018” released by Specified Nonprofit Corporation, Health and Global Policy Institute, the largest percentage of respondents (45%) answered that they “do nothing” to deal with abnormal symptoms associated with menstruation. Therefore, the consumption of familiar and safe plant-derived materials is highly expected to improve women's health. Note that there have been very few reports on the consumption of plant-derived materials for unidentified complaints and other symptoms associated with menstruation in healthy adult women who were not under treatment for or had no history of premenstrual syndrome or premenstrual dysphoric disorder.

Although Apocynum venetum leaf extract has effects on stress reduction, relaxation and sleep improvement, its effects on unidentified complaints, sleep and other symptoms associated with menstruation in healthy adult women are unknown.

### Means to Solve Problems

The inventors carried out studies on the above problems using Apocynum venetum leaf extract and conducted clinical trials to verify the effects on unidentified complaints, sleep and other symptoms associated with menstruation in healthy adult women who were not under treatment for or had no history of premenstrual syndrome or premenstrual dysphoric disorder and demonstrated the effects on unidentified complaints, sleep and other symptoms associated with menstruation for the first time, and as a result, completed the present invention.

Specifically, a composition for improving unidentified complaints before and during menstruation according to one aspect of the present invention comprises Apocynum venetum leaf extract as an active ingredient.

A composition for improving impaired concentration before and during menstruation according to another aspect of the present invention comprises Apocynum venetum leaf extract as an active ingredient.

A composition for improving depression before menstruation according to another aspect of the present invention comprises Apocynum venetum leaf extract as an active ingredient.

A composition for improving decreased motivation for socialization and daily activities before menstruation according to another aspect of the present invention comprises Apocynum venetum leaf extract as an active ingredient.

A composition for improving autonomic nervous system disorders before menstruation according to another aspect of the present invention comprises Apocynum venetum leaf extract as an active ingredient.

A composition for improving pain during menstruation according to another aspect of the present invention comprises Apocynum venetum leaf extract as an active ingredient.

A composition for improving control during menstruation according to another aspect of the present invention comprises Apocynum venetum leaf extract as an active ingredient.

A composition for improving sleep before menstruation according to another aspect of the present invention comprises Apocynum venetum leaf extract as an active ingredient.

### Effects of Invention

As mentioned above, the present invention provides a composition for improving unidentified complaints, depression, impaired concentration, decreased motivation for socialization and daily activities, autonomic nervous system disorders, pain, control and poor sleep quality associated with menstruation.

### DESCRIPTION OF EMBODIMENTS

The embodiment of the present invention will be explained in detail below. Note that the invention can be implemented in many different embodiments and is not limited only to the specific forms described in the embodiment and example shown below.

An embodiment of the present invention is a composition for improving unidentified complaints, depression, impaired concentration, decreased motivation for socialization and daily activities, autonomic nervous system disorders, pain, control and poor sleep quality associated with menstruation.

The Apocynum venetum leaf extract of the present invention is an extract obtained by extracting and concentrating leaves of Apocynum venetum L. in a solvent containing at least one of water, ethanol, hydrous ethanol and an organic solvent, an extract obtained by adsorbing the aforementioned extract to at least one of acrylic, styrene, methacrylic and aromatic synthetic adsorbents and concentrating a fraction eluted with hydrous ethanol with a concentration of 10 to 95%, and an extract obtained by drying the aforementioned extract.

The composition can take the form of a food, a medicine or a cosmetic, and particularly in the case of a food, it can take the form of a food with functional claims or a health food. In the case of food, it can also take the form of a beverage as well as a solid product.

The form of the composition as a food, for example, can be a drink, a candy, a jelly, a gummy candy or other desserts, while the form as a health food or a food with functional claims, for example, can be a tablet, a hard capsule, a soft capsule, a granule, a drink, etc.

The form of the composition as a medicine can be a tablet, a capsule, a pill, a liquid, an emulsion, etc. The method of administration is not limited, but it is preferably in a form that can be administered orally. Various carriers can also be added to the extent that they are pharmaceutically acceptable. The carriers, for example, can be an excipient, a coloring agent, a sweetening agent, a suspending agent, etc.

The content of Apocynum venetum leaf extract in the composition can be adjusted according to the expected intake.

To obtain the effects of the composition in improving unidentified complaints, depression, impaired concentration, decreased motivation for socialization and daily activities, autonomic nervous system disorders, pain, control and poor sleep quality associated with menstruation, the Apocynum venetum leaf extract is preferably 30 mg or more to 150 mg or less, more preferably 35 mg or more to 125 mg or less, and certainly 40 mg to 100 mg per day.

The Apocynum venetum leaf extract of the composition comprises the flavonoid compounds including hyperoside and isoquercitrin as active ingredients, and the total amount of these ingredients is preferably 4% by weight or more, and more preferably in the range of 4% or more to 10% or less by weight to favorably exert effects of the composition.

The composition has effects on improving unidentified complaints, depression, impaired concentration, decreased motivation for socialization and daily activities, autonomic nervous system disorders, pain, control and poor sleep quality associated with menstruation. The range in which these effects can be obtained is also the same as above, as will be clear from the description of the example below.

As mentioned above, the present invention provides a composition for improving unidentified complaints, depression, impaired concentration, decreased motivation for socialization and daily activities, autonomic nervous system disorders, pain, control and poor sleep quality associated with menstruation.

### Example 1

Here, a composition according to the above embodiment was actually produced and its effects were tested. The following is a specific explanation.

(1) Production of Apocynum venetum Leaf Extract

Dried Apocynum venetum leaves were crushed using a lab mixer, and 1 kg of the crushed leaves was extracted with 6 L of 60% ethanol, followed by filtration to obtain an extract. The extracted residue was extracted again with 6 L of 60% ethanol and filtered to obtain an extract. The first and second extracts were mixed, concentrated under reduced pressure at 60° C., adjusted to acidic conditions, passed through synthetic adsorption resin to adsorb the active ingredients, and then rinsed with water to remove sugars and other substances. The extract was then desorbed with hydrous ethanol to collect the fraction containing the active ingredients, concentrated under reduced pressure at 50° C., and dried to obtain a solid product of 95 g.

The liquid chromatographic measurement of the solid product obtained above demonstrated that it contained 2 to 5% by weight of hyperoside and 2 to 58 by weight of isoquercitrin, totaling 4 to 10% by weight.

### (2) Clinical Trials

Tablets containing 25 mg of Apocynum venetum leaf extract per tablet were used in clinical trials. To ensure that placebos had the same appearance as the test food, a food additive formulation containing a coloring agent was used in place of the Apocynum venetum leaf extract.

TABLE 1

| | | | Compounded | |
|---|---|---|---|---|
| Name | Form | Ingredients (per tablet) | amount | |
| Apocynum | Tablet | Apocynum venetum leaf extract | 25.00 | mg |
| venetum | | Starch degradation product | 84.00 | mg |
| leaf | | Crystalline cellulose | 59.50 | mg |
| extract | | Lactose | 22.70 | mg |
| tablet | | Edible fats and oils | 6.30 | mg |
| | | Glycyrrhiza extract | 2.50 | mg |
| | | Guar gum | Trace amount | |
| | | Shellac | Trace amount | |
| | | Glycerol fatty acid ester | Trace amount | |
| | | Carnauba wax | Trace amount | |
| Placebo | Tablet | Starch degradation product | 84.00 | mg |
| tablet | | Crystalline cellulose | 59.50 | mg |
| | | Caramel coloring | 25.00 | mg |
| | | Lactose | 22.70 | mg |
| | | Edible fats and oils | 6.30 | mg |
| | | Glycyrrhiza extract | 2.50 | mg |

| | |
|---|---|
| Guar gum | Trace amount |
| Shellac | Trace amount |
| Glycerol fatty acid ester | Trace amount |
| Carnauba wax | Trace amount |

A randomized, placebo-controlled, double-blind, parallel-group study was conducted in 42 healthy adult women who were not under treatment for or had no history of premenstrual syndrome or premenstrual dysphoric disorder to evaluate the effects on unidentified complaints, sleep and other symptoms associated with menstruation. The subjects consumed the test foods from 7 days after the first menstrual start date until 7 days after the next menstrual start date (one menstrual cycle). The intervention group took 50 mg/day of Apocynum venetum leaf extract, while the placebo group took the same number of placebo tablets.

The Japanese version of the Menstrual Distress Questionnaire (MDQ), which evaluates subjective symptoms related to unidentified complaints associated with menstruation, was used to assess subjective symptoms before and during menstruation before and after intake. The total score for unidentified complaints associated with menstruation and the eight subscales (pain, fluid retention, autonomic nervous system, negative emotions, concentration, behavioral changes, mood elevation and control) were rated on a five-point scale (4=very strongly felt/very strongly symptomatic, 3=strongly felt/strongly symptomatic, 2=somewhat felt/somewhat symptomatic, 1=slightly felt/slightly symptomatic, 0=not felt at all/not at all symptomatic). The higher the score, the stronger the degree of discomfort.

In addition, the MA version of the OSA Sleep Questionnaire was used before and after intake to evaluate how subjects experienced improved sleep. The higher the score, the better the sleep condition.

### Clinical Trial Results

(1) Total MDQ Score (Unidentified Complaints Associated with Menstruation)

The intervention group showed significant improvement over the placebo group in the total MDQ score, which evaluates unidentified complaints associated with menstruation, before and during menstruation after intake (P<0.05).

TABLE 2

| | Intervention | Placebo | Significant |
|---|---|---|---|
| | group | group | difference |
| Before menstruation | 38.9 +/− 16.3 | 50.0 +/− 24.7 | P = 0.016 |
| During menstruation | 42.9 +/− 24.5 | 44.0 +/− 22.7 | P = 0.016 |

### (2) Subscale “Concentration”

The intervention group showed significant improvement over the placebo group in the subscale “concentration,” which evaluates unidentified complaints associated with menstruation, before and during menstruation after intake (P<0.05).

TABLE 3

| | Intervention | Placebo | Significant |
|---|---|---|---|
| | group | group | difference |
| Before menstruation | 8.0 +/− 4.4 | 10.0 +/−6.6 | P = 0.049 |
| During menstruation | 5.5 +/− 1.1 | 8.0 +/−1.0 | P = 0.045 |

### (3) Subscale “Negative Emotions (Depression)”

The intervention group showed significant improvement over the placebo group in the subscale “negative emotions (depression),” which evaluates unidentified complaints associated with menstruation, before menstruation after intake (P<0.05).

TABLE 4

| | Intervention | Placebo | Significant |
|---|---|---|---|
| | group | group | difference |
| Before menstruation | 6.9 +/− 4.8 | 9.1 +/− 6.9 | P = 0.034 |

### (4) Subscale “Behavioral Changes (Motivation for Socialization and Daily Activities)”

The intervention group showed significant improvement over the placebo group in the subscale “behavioral changes (motivation for socialization and daily activities),” which evaluates unidentified complaints associated with menstruation, before menstruation after intake (P<0.05).

TABLE 5

| | Intervention | Placebo | Significant |
|---|---|---|---|
| | group | group | difference |
| Before menstruation | 5.4 +/− 2.8 | 7.1 +/− 3.8 | P = 0.025 |

### (5) Subscale “Autonomic Nervous System”

The intervention group showed significant improvement over the placebo group in the subscale “autonomic nervous system,” which evaluates unidentified complaints associated with menstruation, before menstruation after intake (P<0.05).

TABLE 6

| | Intervention | Placebo | Significant |
|---|---|---|---|
| | group | group | difference |
| Before menstruation | 1.3 +/− 2.1 | 2.5 +/− 2.6 | P = 0.045 |

### (6) Subscale “Pain”

The intervention group showed significant improvement over the placebo group in the subscale “pain,” which evaluates unidentified complaints associated with menstruation, during menstruation after intake (P<0.05).

TABLE 7

| | Intervention | Placebo | Significant |
|---|---|---|---|
| | group | group | difference |
| During menstruation | 7.2 +/− 4.2 | 8.2 +/− 4.8 | P = 0.049 |

### (7) Subscale “Control”

The intervention group showed significant improvement over the placebo group in the subscale “control,” which evaluates unidentified complaints associated with menstruation, during menstruation after intake (P<0.05).

TABLE 8

| | Intervention | Placebo | Significant |
|---|---|---|---|
| | group | group | difference |
| During menstruation | 1.1 +/− 1.6 | 1.9 +/− 2.7 | P = 0.044 |

### (8) Results of MA Version of OSA Sleep Questionnaire

The intervention group showed significant improvement over the placebo group in Factor II, which evaluates falling asleep and staying asleep, before menstruation after intake (P<0.05).

TABLE 9

| | Intervention | Placebo | Significant |
|---|---|---|---|
| | group | group | difference |
| Before menstruation | 18.5 +/− 5.7 | 15.3 +/− 5.1 | P = 0.048 |

As mentioned above, human studies have demonstrated that it has effects on improving unidentified complaints, depression, impaired concentration, decreased motivation for socialization and daily activities, autonomic nervous system disorders, pain, control and poor sleep quality associated with menstruation.

What is claimed is:

1. A composition for improving unidentified complaints before and during menstruation comprising Apocynum venetum leaf extract as an active ingredient.

2. The composition for improving unidentified complaints before and during menstruation according to claim 1, wherein the Apocynum venetum leaf extract comprises 4 to 10% by weight in total of a mixture of flavonoid compounds including hyperoside and isoquercitrin.

3. The composition for improving unidentified complaints before and during menstruation according to claim 1, wherein a daily intake of the Apocynum venetum leaf extract is in the range of 40 mg or more to 100 mg or less.

4. A composition for improving impaired concentration before and during menstruation comprising Apocynum venetum leaf extract as an active ingredient.

5. The composition for improving impaired concentration before and during menstruation according to claim 4, wherein the Apocynum venetum leaf extract comprises 4 to 10% by weight in total of a mixture of flavonoid compounds including hyperoside and isoquercitrin.

6. The composition for improving impaired concentration before and during menstruation according to claim 4, wherein a daily intake of the Apocynum venetum leaf extract is in the range of 40 mg or more to 100 mg or less.

7. A composition for improving depression before menstruation comprising Apocynum venetum leaf extract as an active ingredient.

8. The composition for improving depression before menstruation according to claim 7, wherein the Apocynum venetum leaf extract comprises 4 to 10% by weight in total of a mixture of flavonoid compounds including hyperoside and isoquercitrin.

9. The composition for improving depression before menstruation according to claim 7, wherein a daily intake of the Apocynum venetum leaf extract is in the range of 40 mg or more to 100 mg or less.

10. A composition for improving decreased motivation for socialization and daily activities before menstruation comprising Apocynum venetum leaf extract as an active ingredient.

11. The composition for improving decreased motivation for socialization and daily activities before menstruation according to claim 10, wherein the Apocynum venetum leaf extract comprises 4 to 10% by weight in total of a mixture of flavonoid compounds including hyperoside and isoquercitrin.

12. The composition for improving decreased motivation for socialization and daily activities before menstruation according to claim 10, wherein a daily intake of the Apocynum venetum leaf extract is in the range of 40 mg or more to 100 mg or less.

13. A composition for improving autonomic nervous system disorders before menstruation comprising Apocynum venetum leaf extract as an active ingredient.

14. The composition for improving autonomic nervous system disorders before menstruation according to claim 13, wherein the Apocynum venetum leaf extract comprises 4 to 10% by weight in total of a mixture of flavonoid compounds including hyperoside and isoquercitrin.

15. The composition for improving autonomic nervous system disorders before menstruation according to claim 13, wherein a daily intake of the Apocynum venetum leaf extract is in the range of 40 mg or more to 100 mg or less.

16. A composition for improving pain during menstruation comprising Apocynum venetum leaf extract as an active ingredient.

17. The composition for improving pain during menstruation according to claim 16, wherein the Apocynum venetum leaf extract comprises 4 to 10% by weight in total of a mixture of flavonoid compounds including hyperoside and isoquercitrin.

18. The composition for improving pain during menstruation according to claim 16, wherein a daily intake of the Apocynum venetum leaf extract is in the range of 40 mg or more to 100 mg or less.

19. A composition for improving control during menstruation comprising Apocynum venetum leaf extract as an active ingredient.

20. The composition for improving control during menstruation according to claim 19, wherein the Apocynum venetum leaf extract comprises 4 to 10% by weight in total of a mixture of flavonoid compounds including hyperoside and isoquercitrin.

21. The composition for improving control during menstruation according to claim 19, wherein a daily intake of the Apocynum venetum leaf extract is in the range of 40 mg or more to 100 mg or less.

22. A composition for improving sleep before menstruation comprising Apocynum venetum leaf extract as an active ingredient.

23. The composition for improving sleep before menstruation according to claim 22, wherein the Apocynum venetum leaf extract comprises 4 to 10% by weight in total of a mixture of flavonoid compounds including hyperoside and isoquercitrin.

24. The composition for improving sleep before menstruation according to claim 22, wherein a daily intake of the Apocynum venetum leaf extract is in the range of 40 mg or more to 100 mg or less.
